(19) United States
(12) Patent Application Publication
(10) Pub. No.: US 2017/0252509 A1
(43) Pub. Date: Sep. 7, 2017

(54) MEDICAMENT DELIVERY DEVICE WITH STERILIZING PAD

(71) Applicant: Sanofi, Paris (FR)

(72) Inventor: Michael Caspers, Frankfurt am Main (DE)

(21) Appl. No.: 15/510,176

(22) PCT Filed: Sep. 11, 2015
(86) PCT No.: PCT/EP2015/070873

§ 371 Date: Mar. 9, 2017

(30) Foreign Application Priority Data
Sep. 15, 2014 (EP) 14306422.8

(51) Int. Cl. A61M 5/142

(52) U.S. Cl. CPC A61M 5/14248; A61M 2005/14252; A61M 2005/14264

(57) ABSTRACT

A medicament delivery device comprises a housing containing a medicament delivery mechanism including an injection needle. The housing includes a contact surface having a first adhesive layer. The device further includes a sterilizing pad adhered to the housing. A second adhesive layer is formed proximate the sterilizing pad. A cover is detachably adhered over the sterilizing pad by the second adhesive layer and covers the sterilizing pad.

### CROSS-REFERENCE TO RELATED APPLICATIONS

This application is a U.S. national stage application under 35 USC §371 of International Application No. PCT/EP2015/070873, filed on Sep. 11, 2015, which claims priority to European Patent Application No. 14306422.8, filed on Sep. 15, 2014, the entire contents of which are incorporated herein by reference.

### TECHNICAL FIELD

The present disclosure relates to a device for delivery of medicament to a patient.

### BACKGROUND

A variety of diseases exist that require regular treatment by injection of a medicament. Such injections can be performed by using injection devices. Injection or infusion pumps of the type known as patch pumps for delivering injections of medicament are known in the art. Another type of injection pump that is gaining traction is the bolus injector device. Some bolus injector devices are intended to be used with relatively large volumes of medicament, typically at least 1 ml and maybe a few ml. Injection of such large volumes of medicament can take minutes or even hours. Such high capacity bolus injector devices can be called large volume devices (LVDs). Generally, such devices are operated by the patients themselves, although they may also be operated by medical personnel.

To use a patch pump or bolus injector device such as an LVD, it is first supported on a suitable injection site on a patient's skin and, once installed, injection is initiated by the patient or another person (user). Typically, the initiation is effected by the user operating an electrical switch, which causes a controller to operate the device. Operation includes firstly injecting a needle into the user and then causing the injection of medicament into the user's tissue. Biological medicaments are being increasingly developed which comprise higher viscosity injectable liquids and which are to be administered in larger volumes than long-known liquid medicaments. LVDs for administering such biological medicaments may comprise a pre-filled disposable drug delivery device or, alternatively, a disposable drug delivery device into which a patient or medical personnel must insert a drug cartridge prior to use.

Particularly in the case of patient-operated LVDs which require insertion of a drug cartridge prior to use, the drug delivery process from start to finish can be a complicated multi-step process, including gathering of all of the device components, assembly of the components to produce the LVD ready for drug administration and sterilization of the injection site before the actual process of injecting the drug can even begin. For example, the preparation step includes sourcing a sterilizing liquid, a sterilizing swab to apply the sterilizing liquid, and possibly also a drying swab to dry the injection site of sterilizing liquid. Gathering all these materials is time-consuming and complicated for the patient to remember, intrusive upon his or her daily schedule, and increase the risk that the patient may not correctly perform the drug administration.

There are limitations as to the maximum volume of liquid medicament one injection site can accept within a predetermined amount of time without causing the patient discomfort, pain, inhibiting pharmacokinetics or causing leakage out of the injection site. To avoid complications of such interactions between the drug and the patient's body, such large-volume biological medicaments should not be administered at the same injection site on the patient's body twice or more in succession. Therefore, this is another factor in the medicament administration process which the patient must take into consideration.

### SUMMARY

Certain embodiments provide a medicament delivery device comprising a housing containing a medicament delivery mechanism including an injection needle, the housing including a contact surface having a first adhesive layer, a sterilizing pad adhered to the housing, a second adhesive layer formed proximate the sterilizing pad, and a cover detachably adhered over the sterilizing pad by the second adhesive layer and covering the sterilizing pad.

The sterilizing pad may be disposed on a substrate, and the second adhesive layer may be formed on the substrate. The substrate may be detachably adhered to the housing. This may allow a user to remove and discard the sterilizing pad after use, but before use of the medicament delivery device to administer a medicament. This may provide improved usability, without needing the, possible damp, sterilizing pad to remain on the housing during the medicament delivery process, as well as providing sanitary benefits.

The sterilizing pad may be formed of a compressible material and may be retained in a compressed state by the cover, and expands upon detachment of the cover. This may advantageously allow the pad to project from the substrate when the cover is removed so that only the sterilizing pad, and not the substrate or housing, is wiped across the patient's skin.

The sterilizing pad may comprise an absorbent material impregnated with a sterilizing agent.

The sterilizing pad may include a dye to stain a patient's skin when the sterilizing pad is rubbed against the skin. This may advantageously indicate the area of the patient's skin that has been sterilized, and may also allow a patient to identify a previous injection site to avoid consecutively using the same site.

The sterilizing agent with which the sterilizing pad is impregnated may include a topical anesthetic. This may advantageously locally anesthetize an area of the patient's skin that is sterilized. This may advantageously reduce pain or discomfort that may otherwise be caused by injection of the needle into the patient's skin.

The cover may comprise a cover sheet. The cover and/or the substrate may comprise pull tabs to facilitate their respective detachment from the substrate and the housing.

The second adhesive layer may surround the sterilizing pad. This may advantageously enable the cover to seal against the second adhesive layer completely around the sterilizing pad. This may advantageously seal the sterilizing pad from an ambient environment before removal of the cover. This may advantageously avoid the sterilizing pad drying out and/or becoming contaminated. This may advantageously avoid sterilizing agent from leaking from the sterilizing pad or evaporating or leaching from the sterilizing pad.

The pull tabs may include visual identifiers to indicate to a patient the order in which each should be pulled in correct operation of the medicament delivery device. Such indicators may comprise sequential numbers, letters, or other indicia, graphics or markings. This may make the device easier to use by a patient, and help avoid incorrect use of the device.

The substrate may be adhered to the contact surface of the housing by the first adhesive layer. This may be the same adhesive layer that serves to secure the device to a patient's skin in use. This may advantageously avoid the need for separate adhesive layers to be provided to secure the cover and to adhere the device to a patient, making manufacture simpler and also more cost effective.

The cover may be more weakly adhered to the substrate than the substrate is adhered to the housing. This may advantageously prevent the sterilizing pad being accidentally pulled off housing when removing cover.

The medicament delivery device may further comprise a drying pad disposed on a surface of the housing. This advantageously provides a further component of a medicament administration process integrally with the device, improving usability and simplicity for the patient. This may allow excess sterilizing agent to be removed from a patient's skin before securing the device to a patient.

The substrate may comprise a first substrate, and the medicament delivery device may further comprise a drying pad disposed on a second substrate and wherein the second substrate may be detachably adhered to the housing. This may improve usability, without needing the, possible damp, drying pad to remain on the housing during the medicament delivery process, as well as providing sanitary benefits.

The second substrate may comprise a pull tab to facilitate its detachment from the housing. This may provide ease of use by the patient in removing the drying pad from the device before use.

The second substrate may be interposed between the first substrate and the housing. This may advantageously provide a compact device design using multi-layer construction. Also, the drying pad may be covered by the sterilizing pad making it more unlikely that a patient may use the device incorrectly or in the wrong order.

A third adhesive layer may be provided on the second substrate, and the second substrate may be adhered to the contact surface of the housing by the first adhesive layer, and the first substrate may be adhered to the second substrate by the third adhesive layer.

The drying pad may be formed of a compressible material and may be retained in a compressed state by the first substrate, and expands outwardly from the second substrate upon detachment of the first substrate. This may advantageously cause the drying pad to project from the substrate so that only the drying pad is wiped across a patient's skin, and not the surrounding adhesive on the second substrate.

The cover may be more weakly adhered to the first substrate than the first substrate is adhered to the second substrate, and the first substrate may be more weakly adhered to the second substrate than the second substrate is adhered to the contact surface of the housing. This may advantageously prevent the drying and sterilizing pads being pulled off the housing when removing the cover, and may prevent the drying pad being pulled off the housing when removing the sterilizing pad. This may also help prevent incorrect use of the device by a patient.

Certain embodiments also provide a method of use of a medicament delivery device comprising a housing containing a medicament delivery mechanism including an injection needle, the housing including a contact surface having a first adhesive layer, a sterilizing pad adhered to the housing, a second adhesive layer formed proximate the sterilizing pad, and a cover detachably adhered over the sterilizing pad by the second adhesive layer and covering the sterilizing pad, the method comprising detaching the cover to expose the sterilizing pad, wiping the sterilizing pad across an intended injection site on a patient's skin, securing the device to the patient with the contact surface against the patient's skin over the sterilized injection site, and commencing a medicament delivery process.

Certain embodiments also provide a medicament delivery apparatus comprising medicament delivery device comprising a housing containing a medicament delivery mechanism including an injection needle, a reservoir of medicament for delivery to a patent, a needle insertion mechanism to inject the needle into a patient, the housing including a contact surface having a first adhesive layer for securing the device to a patient's skin, a sterilizing pad impregnated with a sterilizing agent and disposed on a substrate, the substrate detachably adhered to the housing, a second adhesive layer formed on the substrate, and a cover sheet detachably adhered to the substrate by the second adhesive layer and covering the sterilizing pad.

In certain embodiments, the medicament delivery device is easy and simple to use and helps to reduce the risk of incorrect method of use by a user.

### BRIEF DESCRIPTION OF THE DRAWINGS

Embodiments will now be described, by way of example only, with reference to the accompanying drawings, in which:

FIG. 1 shows a side view of a medicament delivery device of a first embodiment;

FIG. 2 shows an exploded perspective view of the medicament delivery device of FIG. 1;

FIG. 3 shows a cross-section of the medicament delivery device along the line X-X shown in FIG. 1;

FIG. 4 shows a side view of a medicament delivery device of a second embodiment;

FIG. 5 shows an exploded perspective view of the medicament delivery device of FIG. 4;

FIG. 6 shows a cross-section of the medicament delivery device along the line Y-Y shown in FIG. 4;

FIG. 7 shows a flow chart of steps of use of the medicament delivery device shown in FIGS. 1 to 3;

FIG. 8 shows an exploded perspective view of a medicament delivery device of a third embodiment; and

FIG. 9 shows an exploded perspective view of a medicament delivery device of a fourth embodiment.

### DETAILED DESCRIPTION

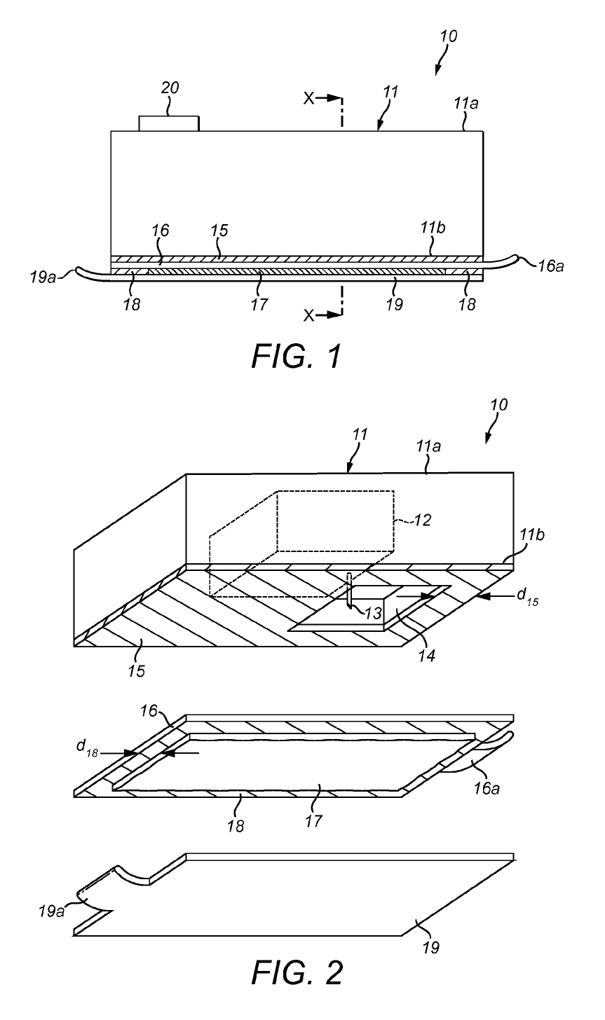

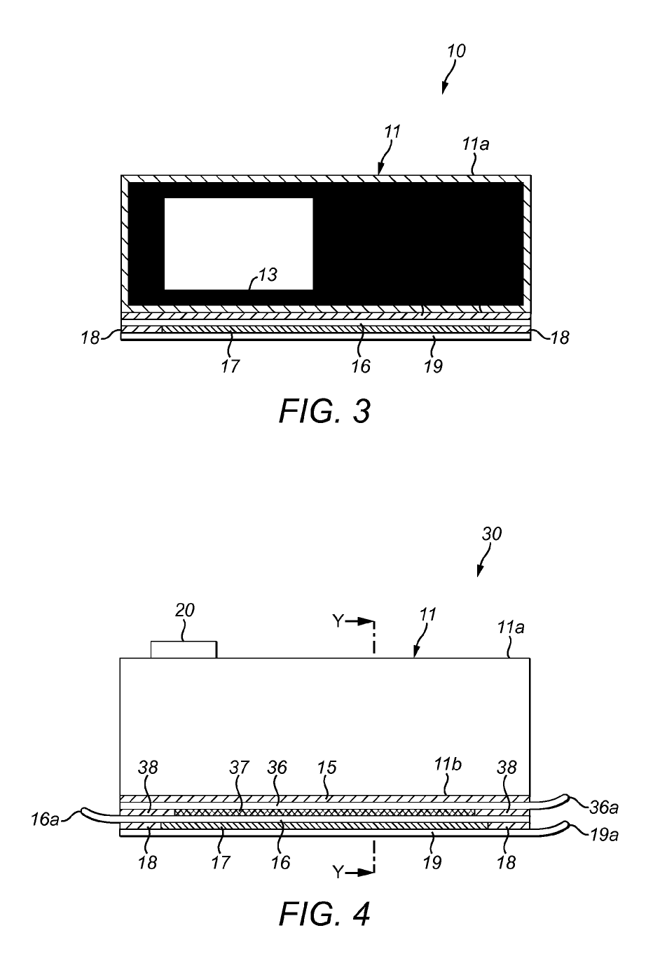

FIGS. 1 to 3 show a medicament delivery device 10, which may be a bolus injector device (hereafter simply referred to as “device 10”) according to a first embodiment which includes a sterilizing swab or pad for a patient to disinfect an intended injection site before commencing a medicament administration process using the device 10. The device 10 comprises a housing 11 containing a medicament delivery mechanism 12. A number of the functional components of the medicament delivery mechanism 12 are omitted for the sake of clarity and brevity, but the medicament delivery mechanism 12 includes a needle 13 for injection of the liquid medicament into a patient's body. The liquid medicament may be provided in a reservoir (not shown) within the medicament delivery mechanism 12 or provided externally of the device 10.

Although not shown in the figures, a medicament delivery mechanism of a device may include one or more of the following components. A controller configured to control operation of the device 10. A needle insertion mechanism to insert the needle 13 into a patient from a retracted position into an engaged position. A needle driver to drive the needle insertion mechanism, for example an electric motor or a spring mechanism. An energy source to power the needle driver. A medicament reservoir containing a supply of medicament to be administered to a patient. The medicament reservoir may, for example, include a cartridge or a vial formed of glass. A plunger maybe provided within the cartridge and plunger driver mechanically coupled to the plunger. The plunger driver may be controllable to move the plunger along the medicament cartridge. The force provided by the plunger causes medicament to be expelled through a medicament delivery aperture in the medicament cartridge and along a medicament delivery tube to the needle 13 to be expelled through the bore of the needle 13. An electrical power source in the form of a battery to power to the controller. The battery may also provide electrical power the plunger driver, if this is an electrically driven device. The battery may also constitute the energy source for the needle driver.

The device 10 generally comprises a housing upper side 11a and a lower side 11b, and in use, the lower side 11b of the housing 11 is intended to be a contact surface that is placed against a patient's skin during a medicament administration process. The contact surface or lower side 11b of the housing 11 includes an aperture 14 through which the needle 13 can project in use. The needle 13 of the medicament delivery mechanism 12 is moveable between a retracted position and an engaged position. In the retracted position the needle 13 is disposed within the housing 11 of the device 10, and in the engaged position, the needle 13 projects from the lower side 11b of the housing 11 through the aperture 14 so as to pierce and inject a patient's skin when the device 10 is attached to a patient.

The lower side 11b of the housing 11 includes a first adhesive layer 15 for adhering the housing 11 to a patient's skin during use. Over the first adhesive layer 15 is a substrate 16 with a sterilizing swab pad 17 bonded to a side of the substrate 16 remote from the housing 11. The sterilizing pad 17 comprises an absorbent material impregnated with a sterilizing agent which may comprise alcohol. The sterilizing pad 17 does not extend to the edges of the substrate 16 and a second adhesive layer 18 is provided around the edge of the substrate 16 surrounding the sterilizing pad 17. A removable cover sheet 19 is disposed over the substrate 16 and sterilizing pad 17 and is held in place by the second adhesive layer 18.

The glue of the first adhesive layer 15 and the material of the adjacent surface of the substrate 16 are configured such that the substrate 16 is not permanently adhered to the lower side 11 b of the housing 11 but is removable therefrom. Similarly, the glue of the second adhesive layer 18 and the material of the adjacent surface of the cover sheet 19 are configured such that the cover sheet 19 is not permanently adhered to the substrate 16 but is removable therefrom.

Use of the device 10 will be described with reference to the flow chart of FIG. 7. A patient peels the cover sheet 19 away from the substrate 16 to expose the sterilizing pad 17 in step S1. In step S2, the patient then uses the sterilizing pad 17 to wipe over the area of their skin which is to serve as the injection site to disinfect the area prior to commencing a medicament administration process. Once the patient has adequately sterilized the injection site, in step S3 they detach the substrate 16 (and with it the attached sterilizing pad 17) from the lower side 11 b of the housing 11 to expose the first adhesive layer 15 and the aperture 14. The patient then adheres the housing 11 to the appropriate part of their body with the aperture 14 placed over the sterilized injection site and the first adhesive layer 15 secures the housing 11 to the patient during the subsequent medicament administration process.

Once the housing 11 is adhered to the patient's body, in step S5 a medicament administration process can be commenced in which the medicament delivery mechanism 12 is actuated to move the needle 13 into the engaged position to pierce the patient's skin, and the medicament is then administered to the patient via the needle 13. The actuation of the medicament delivery mechanism 12 may be manually initiated by the user, for example by pressing a button 20 on the upper side 11a of the housing 11.

It will be appreciated that the device 10 incorporating a sterilizing pad 17 eliminates the need for a user to assemble separate material and equipment in addition to the device 10 in order to perform the medicament administration process, thereby making the procedure simpler and quicker, and less burdensome for the patient.

In order to make use of the device 10 by the patient as simple and user-friendly as possible, the cover sheet 19 and/or the substrate 16 may comprise respective projecting pull tabs 19a, 16a to facilitate a user peeling the cover sheet 19 from the substrate 16 and the substrate 16 from the lower side 11b of the housing 11. The pull tabs 19a, 16a may be numbered or otherwise differently marked for ease of identification by the patient. For example, the cover sheet pull tab 19a may be marked with a “1” and the substrate pull tab 16a may be marked with a “2”. Alternatively, the pull tabs 19a, 16a could be marked with letters, symbols or different colors. Furthermore, to facilitate ease of use, the pull tabs 19a, 16a are preferably spaced apart round the perimeter of the device 10.

It will be appreciated that it is important for correct use of the device 10 that when the patient pulls the cover sheet 19 to remove it, only the cover sheet 19 comes away from the housing 11 by detaching from the second adhesive layer 18 on the substrate 16, and that the substrate 16 does not remain attached to the cover sheet 19 and come away from the lower side 11b of the housing 11 attached to the cover sheet 19. In order to ensure correct operation of the device 10, the second adhesive layer 18 must secure the cover sheet 19 to the substrate 16 with a weaker force than the first adhesive layer 15 secures the substrate 16 to the lower side 11b of the housing 11. This can be achieved in a number of ways. In a first arrangement, the total surface area of first adhesive 15 may be greater than the total surface area of the second adhesive 18. For example, the width d₁₅ of the strip of first adhesive layer 15 around the perimeter of the lower side 11b of the housing 11 may be greater than the width d₁₈ of the strip of the second adhesive layer 18 around the perimeter of the substrate 16. This arrangement is shown in FIG. 2, where it can be seen from the respective shaded areas of the first and second adhesives 15, 18. Also, the lower side 11b of the housing 11 includes a large area of first adhesive layer 15 across its entire surface apart from the region occupied by the aperture 14. In an alternative, or in addition, the first adhesive 15 may be different from the second adhesive 18, such that the first adhesive 15 is stronger than the second adhesive 18.

The sterilizing pad 17 is preferably made from a compressible material and may initially be provided on the substrate 16 in a compressed state and may be retained in the compressed state by the cover sheet 19 adhered to the substrate 16. In such an embodiment, upon detachment of the cover sheet 19, the sterilizing pad 17 expands to project outwardly from the surface of the substrate 16. This ensures that the sterilizing pad 17 makes good contact with the patient's skin in use and avoids the patient rubbing any of the second adhesive layer 18 across their skin.

The sterilizing agent with which the sterilizing pad 17 is impregnated may be a liquid or a gel. An advantage of the sterilizing agent comprising a gel is that the agent would be less susceptible of leaking out from the sterilizing pad 17 between the substrate 16 and the cover sheet 19 during storage of the device 10, and so the device 10 may have a longer shelf or storage life.

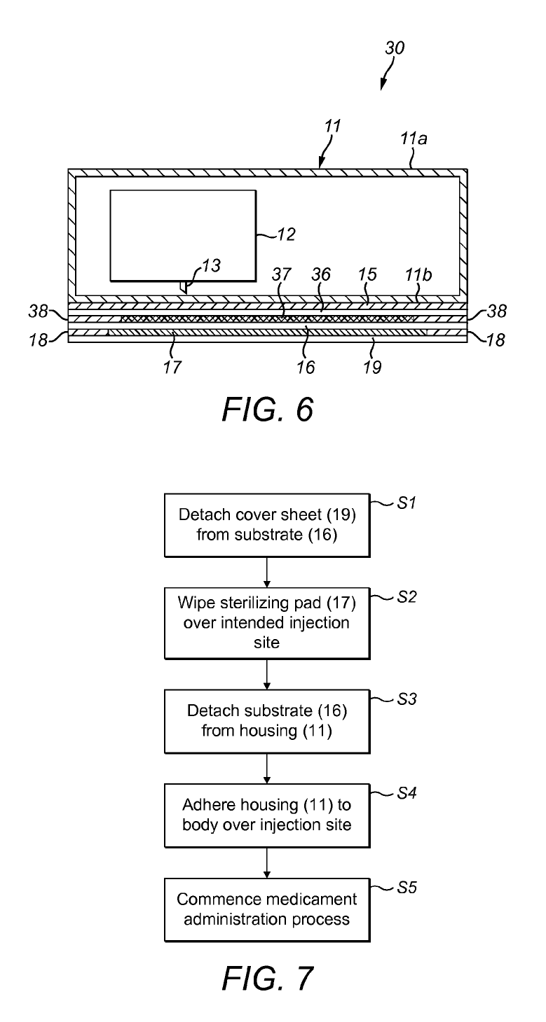

A medicament delivery device 30 of a second embodiment is shown in FIGS. 4 to 6 and is similar to that of the first embodiment, and so like features retain the same reference numerals and a detailed description thereof will not be repeated.

A difference between the device 30 of the second embodiment and that of the first embodiment is that the device 30 of the second embodiment comprises an additional drying layer interposed between the sterilizing pad 17 and a lower side 11b of the housing 11 of the device 30.

The substrate 16 to which the sterilizing pad 17 is bonded comprises a first substrate, and the device 30 further comprises a second substrate 36. The second substrate 36 is provided over the first adhesive layer 15 on the lower side 11b of the housing 11. A drying pad 37 is bonded to a side of the second substrate 36 remote from the device 30. The drying pad 37 comprises a dry absorbent material which may comprise a cotton wool or gauze. The drying pad 37 does not extend to the edges of the second substrate 36 and a third adhesive layer 38 is provided around the edge of the second substrate 36 surrounding the drying pad 37.

The first substrate 16 is adhered to the housing 11 via the second substrate 36, by being provided over the second substrate 36 and held in place thereon by the third adhesive layer 38. As with the first embodiment, the removable cover sheet 19 is disposed over the first substrate 16 and sterilizing pad 17, and is held in place by the second adhesive layer 18.

The glue of the first adhesive layer 15 and the material of the adjacent surface of the second substrate 36 are configured such that the second substrate 36 is not permanently adhered to the lower side 11b of the housing 11 but is removable therefrom. Similarly, the glue of the third adhesive layer 38 and the material of the adjacent surface of the first substrate 16 are configured such that the first substrate 16 is not permanently adhered to the second substrate 36 but is removable therefrom. Also, the glue of the second adhesive layer 18 and the material of the adjacent surface of the cover sheet 19 are configured such that the cover layer 19 is not permanently adhered to the first substrate 16 but is removable therefrom.

In use of the device 30, a patient peels the cover sheet 19 away from the first substrate 16 to expose the sterilizing pad 17. The patient then uses the sterilizing pad 17 to wipe over the area of their skin which is to serve as the injection site to disinfect the area prior to commencing a medicament administration process. Next, the patient peels the first substrate 16 (and with it the attached sterilizing pad 17) away from the second substrate 36 and discards the first substrate 16. This exposes the drying pad 37 which the patient then wipes over the previously disinfected area of their skin to absorb any excess sterilizing agent that may remain on the skin from the sterilizing pad 17. Then, the patient peels the second substrate 36 (and with it the attached drying pad 37) away from the lower side 11b of the housing 11 and discards the second substrate 36. This exposes the first adhesive layer 15 and the aperture 14. The patient then adheres the housing 11 to the appropriate part of their body with the aperture 14 placed over the sterilized and dried injection site and the first adhesive layer 15 secures the housing 11 to the patient during the subsequent medicament administration process, which may be commenced as described above with reference to the first embodiment.

As well as the advantages described above of the incorporated sterilizing pad 17, the device 30 of the second embodiment additionally providing an incorporated drying pad 37 allows the patient to perform an additional preparation step without the need for gathering more separate drying material in addition to the device 30 in order to perform the medicament administration process, thereby making the procedure yet more simple and quicker, and less burdensome for the patient. Allowing the patient to dry the injection site of any excess sterilization agent is also advantageous as it may help ensure a secure bond between the user's skin and the first adhesive layer 15.

As well as the previously-described projecting pull tabs 19a, 16a on the cover sheet 19 and first substrate 16, the second substrate 36 may also include a pull tab 36a to facilitate a user peeling the second substrate 36 from the lower side 11b of the housing 11. As before, all pull tabs 19a, 16a, 36a may be numbered or otherwise differently marked for ease of identification by the patient, for example, by numbers, letters, symbols or different colors, and all pull tabs 19a, 16a, 36a are preferably spaced apart round the perimeter of the device 30.

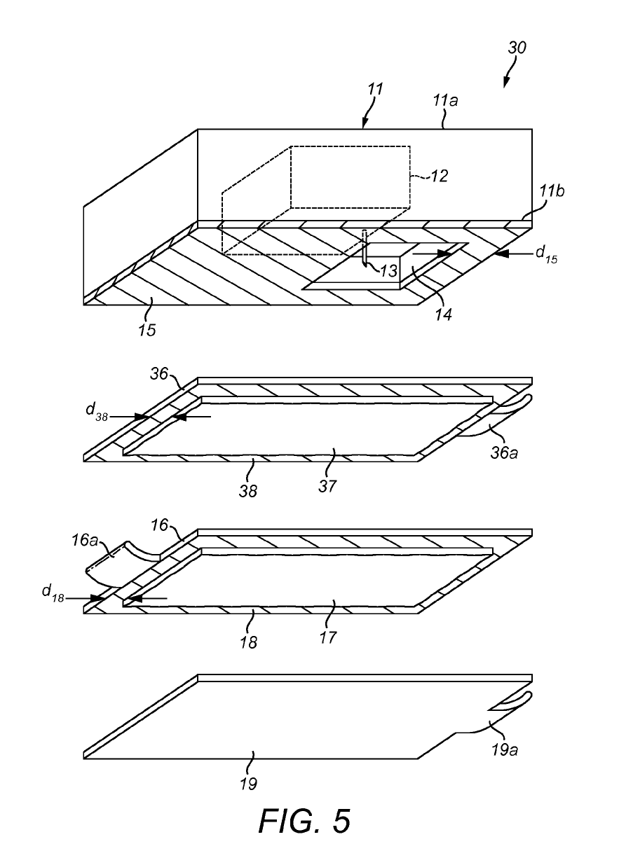

As with the first embodiment, in order to ensure correct use of the device 30, the second adhesive layer 18 must secure the cover sheet 19 to the first substrate 16 with a weaker force than the third adhesive layer 38 secures the first substrate 16 to the second substrate 36. Similarly, the third adhesive layer 38 must secure the first substrate 16 to the second substrate 36 with a weaker force than the first adhesive layer 15 secures the second substrate 36 to the lower side 11b of the housing 11. This can be achieved in a number of ways. In a first arrangement, the total surface area of first adhesive layer 15 may be greater than the total surface area of the third adhesive layer 38, which itself is greater than the total surface area of the second adhesive layer 18. For example, the width d₁₅ of the strip of first adhesive layer 15 around the perimeter of the lower side 11b of the housing 11 may be greater than the width d₃₈ of the strip of the third adhesive layer 38 around the perimeter of the second substrate 36, which in turn is greater than the width d₁₈ of the strip of the second adhesive layer 18 around the perimeter of the first substrate 16. This arrangement is shown in FIG. 5, where it can be seen from the respective shaded areas of the first, third and second adhesive layers 15, 38, 18. Also, the lower side 11b of the housing 11 includes a large area of first adhesive layer 15 across its entire surface apart from the region occupied by the aperture 14. In an alternative, or in addition, the first, third and second adhesive layers 15, 38, 18 may be of different adhesives such that the first adhesive 15 is stronger than the third adhesive 38 which in turn is stronger than the second adhesive 18.

The drying pad 37 is preferably made from a compressible material and may initially be provided on the second substrate 36 in a compressed state and may be retained in the compressed state by the adhered first substrate 16. In such an embodiment, upon detachment of the first substrate 16, the drying pad 37 expands to project outwardly from the surface of the second substrate 36. This ensures that the drying pad 37 makes good contact with the patient's skin in use and avoids the patient rubbing any of the third adhesive layer 38 across their skin.

The material from which the drying pad 37 is made is sterile so as not to contaminate the injection site previously disinfected using the sterilizing pad 17. During manufacture of the device 30, the drying pad may be sterilized and sealed between the first and second substrates 16, 36 with the third adhesive layer to prevent contamination of the drying pad 37 during subsequent manufacture, assembly, transport and storage of the device 30. Alternatively, or in addition, the drying pad 37 may be impregnated with a dry sterilizing agent to maintain the sterility of the drying pad 37 and avoid contamination of the injection site in use. This may provide the additional advantage of avoiding the patient contaminating the injection site, which otherwise may occur if a user was to dry the injection site with a paper towel, cloth or other material which may not be sufficiently clean and sterile.

The sterilizing pad 17 of the first and second embodiments comprises an absorbent material impregnated with a sterilizing agent. However, the invention is not intended to be limited to such a configuration of sterilizing pad and may alternatively include a layer of solid sterilizing material formed on the substrate, such that the entire pad comprises the sterilizing material. Such an embodiment may provide an advantage of simple and therefore cost-effective manufacture, as a layer of sterilizing material may be formed, for example printed, on a substrate without the need for a separate step of impregnating an absorbent carrier material with the desired sterilizing agent. Such an embodiment may also provide an advantage of remaining stable for extended periods of time providing longer shelf or storage life.

In additional embodiments, the sterilizing pad 17 and/or the sterilizing agent may include a dye to stain a patient's skin so as to visually identify an area that has been sterilized. This is may provide an advantage that the patient can easily identify the area of skin that has been sterilized and ensure the device is accurately positioned for the medicament administration process. In addition, the dye may be used as an indicator of which area on the patient's skin was last used as an injection site and help the patient avoid using the same site in consecutive medicament administration processes, which may, for some medicaments, be inadvisable. The dye used may be configured to remain visible on the patient's skin for a pre-determined period of time, or predetermined number of washes of the skin, to correspond to the particular frequency of medicament administration. For example, if the medicament in question is intended for once weekly administration, the dye may be designed to stain the patient's skin for seven or eight days. This way, only the most immediately recent injection site would be visible to the patient and to avoid confusion as to which injection site was last used.

The sterilizing agent with which the sterilizing pad 17 is impregnated, may optionally include a topical anesthetic to locally anesthetize the area of skin that is sterilized. This may beneficially reduce pain or discomfort that may otherwise be caused by injection of the needle into the patient's skin.

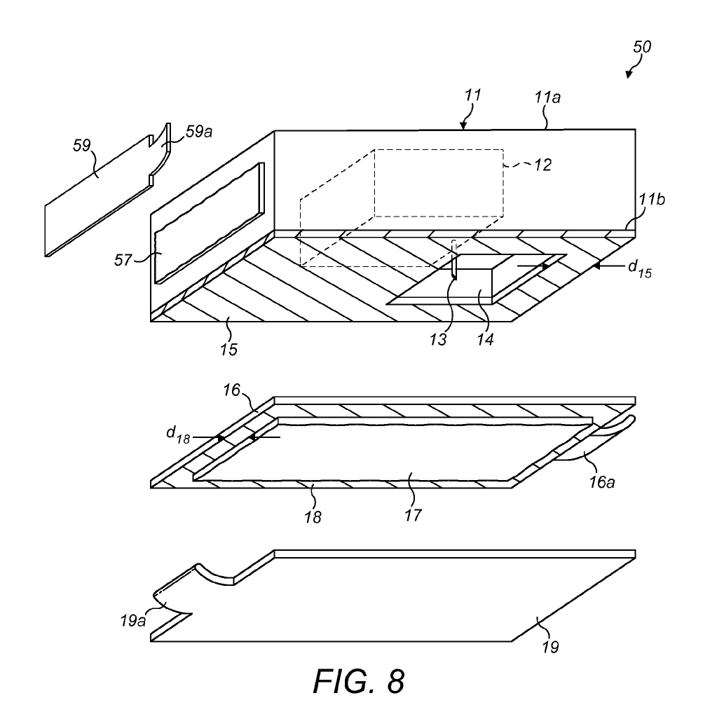

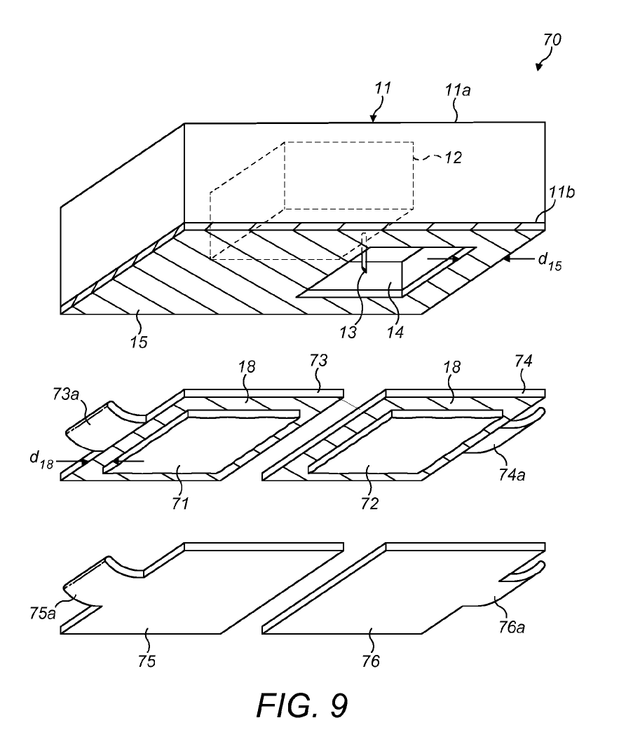

In the second embodiment, the sterilizing pad 17 is provided over the drying pad 37 so that the drying pad 37 is interposed between the sterilizing pad 17 and the housing 11. However, a drying pad may be provided separately to the sterilizing pad. For example, such an alternative third embodiment 50 is shown in FIG. 8 and is similar to that of the first embodiment. However, the device 50 of the third embodiment includes an additional drying pad 57 provided on another face of the housing, for example as shown on the end face, although it may be provided on any other face of the housing. In such an embodiment, the drying pad 57 may be provided on a substrate (not shown) or may be provided directly on the housing, as shown in FIG. 8. If provided on a substrate, the drying pad 57 may be detachable from the housing. Furthermore, the drying pad may include its own cover sheet 59 for covering the drying pad before use, which may advantageously maintain the sterility of the drying pad 57. The cover sheet 59 may include a tab 59a for ease of removal of the cover sheet 59.

A device 70 of a yet further alternative, fourth embodiment, may be similar to the device 10 of the first embodiment, although a sterilizing pad 71 and a drying pad 72 may be provided on respective substrates 73, 74 side by side on the lower side 11b of the housing. Each substrate 73, 74 may include a tab 73a, 74a to facilitate detachment of the substrate 73, 74 from the lower side 11b of the housing 11. Each substrate 73, 74 may include its own cover sheet 75, 76. Each cover sheet 75, 76 may include a tab 75a, 76a to facilitate detachment of the cover sheet 75, 76 from the substrate 73, 74. Both substrates may include an adhesive layer 18 around the sterilizing/drying pad 71, 72 to retain the cover sheets 75, 76 in place before use. A patient may first remove the cover sheet 75 of the sterilizing pad 71 and use the sterilizing pad 71 to sterilize the injection site, and then remove the sterilizing pad substrate 73 from the housing 11. The patient may then remove the cover sheet 76 for the drying pad 72 and dry the sterilized injection site, and then remove the drying pad 72 from the housing 11, leaving the housing 11 with exposed adhesive layer on its lower side 11b for attachment to the patient's skin for a medicament administration process to commence.

In the first and second embodiments, the first adhesive layer 15 is provided to both secure the first or second substrate 16, 36 to the housing 11, but also subsequently secures the housing 11 to the patient's skin in use of the device 10, 30. To achieve this, the first adhesive may more securely attach the housing to the patient's skin than it retains the first or second substrate 16, 36 to the housing 11. This may be achieved by the first or second substrates 16, 36 being made of a material that detachably bonds to the first adhesive layer, for example a waxed paper or plastic material. Alternatively, a backing of the first or second substrates 16, 36, that is the side remote from the sterilizing pad 17 or drying pad 37, may be coated with a material that detachably bonds to the first adhesive layer, for example a waxed or plastic coating.

Notwithstanding the above, the invention is not intended to be limited to embodiments in which a first adhesive layer 15 on the contact surface/lower side 11b of the housing 11 both secures the housing 11 to the patient's skin in use and also detachably adheres the first or second substrates 16, 36 to the housing 11. In a further exemplary embodiment, the lower side 11b of the housing may be provided with a first adhesive layer which may serve to adhere the first or second substrates 16, 36 to the housing 11, and yet may include a separate skin-adhesive layer exclusively or primarily for attaching the housing 11 to a patient's skin during a medicament administration process. In such an exemplary embodiment, the first adhesive layer may be provided in a strip around the perimeter of the lower side 11b of the housing 11, and the skin-adhesive layer may be provided as a patch on the lower side 11b of the housing 11 within the perimeter of the first adhesive layer. In such an exemplary embodiment, the backing surface of the first or second substrates 16, 36 may include different areas of material, or different coatings, to adhere with different strengths to the first adhesive layer and the skin-adhesive layer. Alternatively, or in addition, the adhesives of the first adhesive layer and second adhesive layer may be different, with different properties. For example, the skin-adhesive layer may bond to skin with more strength than it bonds to the first or second substrates, and/or the first adhesive layer may bond to the first and second substrates with more strength than it bonds to skin. In such an embodiment, it may be advantageous that the first substrate is more strongly adhered to the housing by the first adhesive layer, or by both the first adhesive layer and the skin-adhesive layer, than the cover sheet is adhered to the first substrate. Similarly, in an embodiment including a drying pad, it may be advantageous that the second substrate is more strongly adhered to the housing by the first adhesive layer, or by both the first adhesive layer and the skin-adhesive layer, than the first substrate is adhered to the second substrate, and also that the first substrate is more strongly adhered to the second substrate than the cover sheet is adhered to the first substrate.

It will be appreciated that the thickness of the first, second and third adhesive layers 15, 18, 38, the first and second substrates 16, 36, the sterilizing pad 17, the drying pad 37 and the cover sheet 19 are not shown to scale in FIGS. 1 to 6 but are exaggerated for clarity of illustration.

The lower side 11b of the housing 11 is shown as a substantially planar contact surface, although the invention is not limited to such a configuration in alternative embodiments, the contact surface may be curved or otherwise contoured. Such an embodiment may be advantageous as it may enable the device 10 to fit to the contours of a patient's body to which it is intended to be secured, for example the thigh or torso.

Although the first and second substrates 16, 36 are shown and described as covering the entire surface area of the lower side 11b of the housing 11, the invention is not intended to be limited to such configuration and the first and second substrates 16, 36 may alternatively only partially cover the lower side 11b of the housing 11.

In addition to the above, the invention is not limited to devices in which the sterilizing pad and/or drying pad are provided on the lower side 11b of the housing 11, or on the surface of the device that is intended to be secured against a patient's skin. The device may include a sterilizing pad and/or drying pad on any other surface of the device, for example the side or upper faces.

Devices 10, 30, 50, 70 are described as having a cover sheet 19, 59, 75, 76 to cover the sterilizing pad 17, 71 and/or drying pad 57, 72 before use. However, the invention is not limited to devices which include such a cover sheet 19, 59, 75, 76 and other configurations of cover may be provided to cover the sterilizing pad 17, 71 and/or drying pad 37, 57, 72 prior to use. The device may be provided in a packaging (not shown) with the substrate and/or housing surface secured to a surface of the packaging, which thereby covers the sterilizing pad 17, 71 and/or drying pad 37, 57, 72. A surface of the packaging may thereby serve as a cover layer in place of a cover sheet. When a patient then removes the device 10, 30, 50, 70 from the packaging, the sterilizing pad 17, 71 and/or drying pad 37, 57, 72 is then exposed ready for use by a patient. This may advantageously reduce the number of steps of the process for a patient, as a separate cover sheet removal step is not required. This provides benefits of usability for the patient. It also would reduce waste, as the packaging would also function as the cover sheet, without the need for a separate cover sheet(s) 19, 59, 75, 76.

In devices 10, 30, 50, 70 shown and described, the sterilizing pads 17, 71 are described as being impregnated with a sterilizing agent. However, the invention is not limited to such devices with a pre-impregnated sterilizing pad and in alternative embodiments, the sterilizing pad could comprise an absorbent pad which, prior to use, may be impregnated with a sterilizing agent by a patient, for example by being soaked in a sterilizing agent or dipped into, or dabbed onto, a source of sterilizing agent. Such source of sterilizing agent may comprise a container of sterilizing material, which may be a liquid or a gel, or a solid sterilizing agent that is loaded onto the sterilizing pad by the pad being rubbed onto the sterilizing material. Such a source of sterilizing agent may be supplied with the device 10, 30, 50, 70, for example together in a packaging of the device. Alternatively, the container could be formed on or bonded to a cover sheet of the device.

It will be appreciated that the inventive concept of medicament delivery devices may be applicable to LVDs. However, the invention is not intended to be limited to this particular type of medicament delivery device and is intended to cover alternative types of medicament delivery devices which function in contact with a patient's skin, such as, for example, patch pumps and infusion pumps.

The medicament delivery device includes a needle to pierce a patient's skin as part of the process of injecting a medicament through a patient's skin into their body. Such devices include, for example, patch pumps and infusion devices in which the medicament is delivered into the patient's tissue. The embodiments particularly suited to bolus injections, but the injection device may instead be of the basal type.

In certain embodiments, devices may include a hollow needle through which the medicament is delivered, or a solid needle, such as in trocar devices, in which a solid needle or obturator pierces the skin and a hollow tube or cannula is subsequently inserted into the pierced hole and through which the medicament is subsequently delivered to the patient. In trocar devices, the solid needle or obturator does not remain in the patient's skin during medicament delivery.

The medicament delivery mechanism of the embodiments may take any suitable form. It may for instance include an electric motor and a gear mechanism that causes insertion of the needle 13 into the user. It may alternatively be a mechanical spring based mechanism. In this case the needle 13 driving energy source is a preloaded spring, and a needle insertion mechanism driver may be a spring release mechanism that causes force from the spring to be communicated to a needle insertion mechanism thereby to insert the needle 13 into the patient.

An insertion mechanism for inserting the needle may take any suitable form. It may be a mechanical spring based mechanism. Alternatively, the insertion element mechanism may for instance include an electric motor and a gear mechanism that causes insertion of the insertion element into the user. In alternative embodiments, a needle insertion mechanism driver may be a gas or fluid pressure operated mechanism, in which case the needle driving energy source is either a reservoir of pressurized gas or a chemical system in which two or more chemicals are mixed together to produce gas or fluid pressure.

The sterilizing pad 17 may comprise an absorbent pad impregnated with a suitable sterilizing agent. Exemplary sterilizing agents include, but are not limited to, isopropanol, isopropyl alcohol, isopropyl alcohol as a dissolution, for example isopropyl alcohol as a 70% dissolution, tincture of iodine, hydrogen peroxide, chloramine T, alcohol (e.g. ethanol, 1-propanol), phenols, nitrogen compounds, chlorhexidin, and/or detergents.

The drying pad 37 may comprise a dry absorbent material which may comprise a cotton wool or gauze. However, the drying pad 37 may comprise any suitable material and may include, but is not limited to, gauze sponge, cotton, cellulose, rayon, or other porous filter material.

The device is configured to deliver the medicament subcutaneously, although it may instead be configured for intradermal injection, for instance using a microneedle, or for injection in some other manner.

The bolus injector device may be of the type known as a Large Volume Device (LVD). An LVD injection device is configured to dispense a relatively large dose of medicament, in particular at least 1 ml and typically up to 2.5 ml, but possibly up to 10 ml.

The bolus injector device is configured to deliver a bolus of the respective medicament to bring a volume of the medicament into a patient's body within a predetermined time. The injection rate, however, may not be critical, i.e. tight control may not be necessary. However, there may be an upper (physiological) limit to the delivery rate in order to avoid damage to the tissue surrounding the delivery site. The time taken to deliver a bolus dose of medicament may be between a few minutes and many hours depending on a number of factors including the quantity (volume) of medicament, the viscosity of the medicament and the nature of the injection site at which the injection device is intended to be used.

From a user or Health Care Professional perspective, it is desirable for an injection device to be configured to minimally impact the patient's lifestyle and schedule, providing the patient with minimal reminder of his or her disease between the injections. The treatment schedule for therapies is usually intermittent, i.e. may be one injection per week, one injection every other week, or one per month. Therefore, the patient usually has no routine in dealing with his or her disease, and hence has minimal routine/experience in performing the required injections. Thus, configuration of the injection device to simplify its operation by patients is highly desirable.

Because it is intended for bolus operation, the configuration of the injection device is quite different compared to an injection device that is intended to be used for basal operation. Also, its use is quite different. For instance, a basal type insulin pump generally is relatively expensive as it includes many sophisticated diabetes specific features like programmable delivery rate profiles, bolus calculators etc. Further, the connection to the body via an infusion set allows the patient to handle and manipulate the pump in his/her field of view while the therapy is ongoing. Further, diabetes patients usually have a routine in setting-up the infusion set, connecting and operating the pump, and disconnecting the pump temporarily for events like taking a shower so not to expose the pump to water. In contrast, the bolus injector devices described above can be relatively simple and inexpensive devices. They may be provided as single-use devices, which cannot be recharged with medicament, which further reduces complexity and cost. The term “drug” or “medicament”, as used herein, means a pharmaceutical formulation containing at least one pharmaceutically active compound. In some embodiments, the pharmaceutically active compound can have a molecular weight up to 1500 Da or may include a peptide, a protein, a polysaccharide, a vaccine, a DNA molecule, an RNA molecule, an enzyme, an antibody or a fragment thereof, a hormone or an oligonucleotide, or a mixture of the above-mentioned pharmaceutically active compound. Various types or subtypes of compounds are also contemplated. For example, RNA may include RNAi, siRNA, or miRNA. In other embodiments, the pharmaceutically active compound can be useful for the treatment or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis or rheumatoid arthritis. In some embodiments, the pharmaceutically active compound can comprise at least one peptide for the treatment or prophylaxis of diabetes mellitus or complications associated with diabetes mellitus such as diabetic retinopathy. The pharmaceutically active compound can also comprise at least one human insulin or a human insulin analogue or derivative, glucagon-like peptide (GLP-1) or an analogue or derivative thereof, or exendin-3 or exendin-4 or an analogue or derivative of exendin-3 or exendin-4 or a pharmaceutically acceptable salt or solvate thereof.

Insulin analogues can include, for example, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives can include, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(ω-carboxyhepta-decanoyl) human insulin.

Exendin-4 can include, for example, Exendin-4(1-39).

Hormones can include, for example, hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, or Goserelin.

A polysaccharide can include, for example, a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies can include generally globular plasma proteins (˜150 kDa) that are also known as immunoglobulins which share a basic structure. As they can have sugar chains added to amino acid residues, they may also be classified as glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that can include four polypeptide chains; two heavy chains and two light chains connected by disulfide bonds between cysteine residues. Each heavy chain can be about 440 amino acids long; each light chain can be about 220 amino acids long. Heavy and light chains may each contain intra-chain disulfide bonds which stabilize their folding. Each chain is composed of structural domains called Ig domains. These domains typically contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids.

Each heavy chain has two regions, the constant region (CH) and the variable region (VH). In one species, the constant region is essentially identical in all antibodies of the same isotype, but differs in antibodies of different isotypes. Heavy chains λ, α and δ have a constant region composed of three tandem Ig domains, and a hinge region for added flexibility; heavy chains p and E have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of antibodies can be similar, the unique property of a given antibody is determined by the variable (V) regions, as detailed above. More specifically, variable loops, often three on the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is usually the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their inter-chain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H inter-chain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion. Pharmaceutically acceptable solvates are for example hydrates.

In some embodiments, medicaments of various viscosities can be injected. For example, viscosity could range from about 3 to about 50 cP. In other embodiments, viscosity could be less than about 3 cP or greater than about 50 cP. Injection can further include delivering a medicament to a sub-cutaneous, an intra-muscular, or a transdermal location within a patient's body. The medicament can be in the form of a liquid, gel, slurry, suspension, particle, powder, or other type.

Typical injection volumes can range from about 1 mL to about 10 mL. Rates of injection may be about 0.5 mL/min, about 0.2 mL/min, or about 0.1 mL/min. Such injection profiles may be generally constant in flow rate, generally continuous in duration, or both generally constant and generally continuous. These injections can also occur in a single step of administration. Such injection profiles may be referred to as bolus injections.

Delivery devices functioning with such medicaments may utilize a needle, cannula, or other injection element configured to deliver a medicament to the patient. Such an injection element may, for example, have an external size or diameter of 27 G or less. Further, the injection element could be rigid, flexible, and formed using a range of one or more materials. And in some embodiments, the injection element may include two or more components. For example, a rigid trocar may operate in conjunction with a flexible cannula. Initially, both the trocar and cannula may move together to pierce the skin. The trocar may then retract while the cannula remains at least partially within the target tissue. Later, the cannula may separately retract into the delivery device.

1-14. (canceled)

15. A medicament delivery device comprising:
a housing containing a medicament delivery mechanism including an injection needle, the housing including a contact surface having a first adhesive layer;
a sterilizing pad adhered to the housing;
a second adhesive layer formed proximate the sterilizing pad; and
a cover detachably adhered over the sterilizing pad by the second adhesive layer and covering the sterilizing pad.

16. The medicament delivery device according to claim 15, wherein the sterilizing pad is disposed on a substrate, and the second adhesive layer is formed on the substrate.

17. The medicament delivery device according to claim 16, wherein the substrate is detachably adhered to the housing.

18. The medicament delivery device according to claim 17, wherein the substrate is adhered to the contact surface of the housing by the first adhesive layer.

19. The medicament delivery device according to claim 17, wherein the cover is more weakly adhered to the substrate than the substrate is adhered to the housing.

20. The medicament delivery device according to claim 17, wherein:
the substrate comprises a first substrate,
the medicament delivery device further comprises a drying pad disposed on a second substrate, and
the second substrate is detachably adhered to the housing.

21. The medicament delivery device according to claim 20, wherein the second substrate is interposed between the first substrate and the housing.

22. The medicament delivery device according to claim 21, wherein:
a third adhesive layer is provided on the second substrate,
the second substrate is adhered to the contact surface of the housing by the first adhesive layer, and
the first substrate is adhered to the second substrate by the third adhesive layer.

23. The medicament delivery device according to claim 22, wherein the drying pad is formed of a compressible material, is retained in a compressed state by the first substrate, and is configured to expand outwardly from the second substrate upon detachment of the first substrate.

24. The medicament delivery device according to claim 22, wherein the cover is more weakly adhered to the first substrate than the first substrate is adhered to the second substrate, and the first substrate is more weakly adhered to the second substrate than the second substrate is adhered to the contact surface of the housing.

25. The medicament delivery device according to claim 15, wherein the sterilizing pad is formed of a compressible material, is retained in a compressed state by the cover, and is configured to expand upon detachment of the cover.

26. The medicament delivery device according to claim 15, wherein the sterilizing pad comprises an absorbent material impregnated with a sterilizing agent.

27. The medicament delivery device according to claim 15, further comprising a drying pad disposed on a surface of the housing.

28. The medicament delivery device according to claim 15, wherein the sterilizing pad includes a dye to stain skin of a patient when the sterilizing pad is rubbed against the skin.

29. A method comprising:
wiping a sterilizing pad of a medicament delivery device across an injection site on a patient;
securing the medicament delivery device to the injection site with a contact surface of the medicament delivery device against skin of the patient; and then
commencing delivery of medicament from the medicament delivery device.

30. The method of claim 29, further comprising detaching a cover from a housing of the medicament delivery device to expose the sterilizing pad of the medicament delivery device before wiping the sterilizing pad across the injection site.

31. The method of claim 29, further comprising detaching a cover from a housing of the medicament delivery device to expose the contact surface of the medicament delivery device, after sterilizing the injection site and before securing the medicament delivery device to the injection site.

32. The method of claim 29, further comprising wiping a drying pad of the medicament delivery device across the injection site to remove excess sterilizing agent.

33. The method of claim 32, further comprising detaching a cover from a housing of the medicament delivery device to expose a drying pad before wiping the excess sterilization agent from the injection site.

34. The method of claim 29, wherein securing the device to the injection site comprises securing a housing of the medicament delivery device to the injection site, and the sterilizing pad is attached to the housing and overlies the contact surface.
